(19) United States
(12) Patent Application Publication
(10) Pub. No.: US 2023/0117606 A1
(43) Pub. Date: Apr. 20, 2023

(54) COMPOSITIONS AND METHODS FOR THE TREATMENT OF INTRACELLULAR BACTERIAL INFECTIONS

(71) Applicant: Endolytix Technology, Inc., Beverly, MA (US)

(72) Inventors: Jason HOLDER, Swampscott, MA (US); Gary P. MAGNANT, Topsfield, MA (US)

(21) Appl. No.: 17/909,694

(22) PCT Filed: Mar. 8, 2021
(86) PCT No.: PCT/US2021/021410

§ 371 Date: Sep. 6, 2022

Related U.S. Application Data
(60) Provisional application No. 62/986,595, filed on Mar. 6, 2020.

(51) Int. Cl. A61K 38/47; A61K 9/127; A61K 31/175; A61K 31/47; A61K 31/454; A61K 31/4965; A61K 31/4409; A61K 31/7052; A61K 31/133; A61K 31/496; A61K 31/7036; A61P 31/04; A61K 35/76; C12N 7/00; A61K 47/69; A61K 47/68; A61K 47/54; A61K 47/64

(52) U.S. Cl. CPC A61K 38/47; A61K 9/127; A61K 31/175; A61K 31/47; A61K 31/454; A61K 31/4965; A61K 31/4409; A61K 31/7052; A61K 31/133; A61K 31/496; A61K 31/7036; A61P 31/04; A61K 35/76; C12N 7/00; C12Y 302/01017; C12Y 302/01001; C12Y 302/01068; A61K 47/6911; A61K 47/6849; A61K 47/548; A61K 47/64; C12N 2795/00051; C12N 2795/00032

(57) ABSTRACT

The present disclosure features compositions and methods for the treatment of bacterial infections, such as bacterial infections caused by bacterial cells residing within a host cell (e.g., a mammalian cell, e.g., immune cell, e.g., macrophage or dendritic cell). The compositions and methods include delivering antimicrobial agents to specifically target the intracellular compartment (endosome, phagosome, lysosome, or cytosol) in which the bacterial cell resides.

### BACKGROUND

Bacterial pathogens are a leading cause of infectious disease. Many bacteria are successfully detected by the human immune system and are rapidly cleared before onset of infection. However, a number of bacterial pathogens evade the host immune system by residing within a host cell. These intracellular bacteria have evolved diverse immune evasion techniques by residing and multiplying within host cells, such as immune cells (e.g., macrophages or dendritic cells), and the correct intracellular compartment (e.g., endosome, phagosome, lysosome, or cytosol) within the host cells. Bacterial infections that propagate within a host cell often present a difficult treatment barrier due to lack of accessibility of the subcellular location of the infection. While certain anti-bacterial compositions may treat the infection (e.g., in vitro), delivering the treatment to the correct subcellular location in which the bacteria reside has proved to be a challenging endeavor.

One group of challenging intracellular bacterial infections is caused by Mycobacteria. Mycobacteria are actinobacteria that are denoted by a thick cell wall that is rich in mycolic acids. Mycobacteria contain an envelope that contains a cell membrane composed of a lipid bilayer and cell wall that includes a peptidoglycan layer and an arabinogalactan layer, and outer membrane that contains a hydrophobic mycolate layer. Many Mycobacteria also contain an outer capsule composed of polysaccharides, such as D-glucan, D-arabino-D-mannan, and D-mannan. This complex cell envelope contributes to the hardiness of the Mycobacteria and is particularly difficult to penetrate and destroy. Pathogenic Mycobacteria are often partitioned into two groups: M. tuberculosis and non-tuberculosis Mycobacteria (NTM). In contrast to tuberculosis, person-to-person transmission of NTM is rare. Nonetheless, the number of NTM infections is a growing health concern, particularly in people with lung disease.

Improved compositions and methods for targeting and treating intracellular bacterial infections, such as those caused by Mycobacteria, are needed.

### SUMMARY OF THE INVENTION

In one aspect, the invention features a method of delivering a bacteriophage to a targeted intracellular compartment in a professional antigen presenting cell (e.g., macrophage or a dendritic cell) in a subject. The targeted intracellular compartment may include a bacterial cell (e.g., mycobacterial cell). The method includes administering to the subject a composition that includes a supramolecular structure containing a bacteriophage. Following the administering step, the bacteriophage is delivered to the targeted intracellular compartment. The supramolecular structure may further include a targeting moiety.

In another aspect, the invention features a method of delivering a bacteriophage to a targeted intracellular compartment in a professional antigen presenting cell (e.g., macrophage or a dendritic cell) in a subject. The targeted intracellular compartment may include a bacterial cell (e.g., mycobacterial cell). The method includes administering to the subject a composition that includes a supramolecular structure that includes a targeting moiety and a cargo that includes a bacteriophage. Following the administering step, the bacteriophage is delivered to the targeted intracellular compartment.

In another aspect, the invention features a method of treating an intracellular bacterial infection in a subject caused by a bacterial cell. The method includes administering a composition that includes a supramolecular structure and a cargo that includes a bacteriophage. The composition may be administered to the subject in an amount and for a duration sufficient to treat the bacterial infection. The supramolecular structure may further include a targeting moiety.

In another aspect, the invention features a method of treating an intracellular bacterial infection in a subject caused by a bacterial cell. The method includes administering a composition that includes a supramolecular structure that includes a targeting moiety and a cargo that includes a bacteriophage. The composition may be administered to the subject in an amount and for a duration sufficient to treat the bacterial infection.

In another aspect, the invention features a composition that includes a supramolecular structure and a cargo that includes a bacteriophage. The supramolecular structure may further include a targeting moiety.

In another aspect, the invention features a composition that includes a supramolecular structure having a targeting moiety and a cargo that includes a bacteriophage.

In some embodiments of any of the above aspects, the Z-average mean particle diameter of the supramolecular structure is from about 75 nm to about 750 nm (e.g., from about 250 nm to about 750 nm, or from about 75 nm to about 250 nm). Preferably, when the supramolecular structure is an LNP or micelle, the Z-average mean particle diameter is from about 75 nm to about 250 nm. Preferably, when the supramolecular structure is a vesicle (e.g., a liposome), the Z-average mean particle diameter is from about 250 nm to about 750 nm. Non-limiting examples of the Z-average mean particle diameters include from about 75 nm to about 100 nm, from 75 nm to about 85 nm, e.g., about 80 nm, e.g., from about 80 nm to about 140 nm, from about 90 nm to about 130 nm, or from about 110 nm to about 130 nm, e.g., about 120 nm, e.g., from about 200 nm to about 300 nm, e.g., from about 250 nm to about 300 nm, from about 260 nm to about 290 nm, from about 260 nm to about 280 nm, from about 265 nm to about 275 nm, e.g., about 270 nm, e.g., from about 300 nm to about 400 nm, from about 400 nm to about 600 nm, e.g., from about 450 nm to about 550 nm, from about 475 nm to about 525 nm, from about 480 nm to about 520 nm, from about 490 nm to about 510 nm, from about 495 nm to about 505 nm; e.g., about 500 nm, e.g., about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, about 500 nm, about 505 nm, about 510 nm, about 515 nm, about 520 nm, about 525 nm, about 530 nm, about 535 nm, about 540 nm, about 545 nm, about 550 nm, about 555 nm, about 560 nm, about 565 nm, about 570 nm, about 575 nm, about 580 nm, about 585 nm, about 590 nm, about 595 nm, about 600 nm, about 605 nm, about 610 nm, about 615 nm, about 620 nm, about 625 nm, about 630 nm, about 635 nm, about 640 nm, about 645 nm, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, or about 750 nm. In particular embodiments, the Z-average mean particle diameter of the supramolecular structure may be from about 75 nm to about 150 nm. In some embodiments, the Z-average mean particle diameter of the supramolecular structure is about 80 nm, about 270 nm, or about 500 nm.

In some embodiments of any of the above aspects, the bacterial cell is, or the bacteriophage is capable of infecting, a Mycobacterium, Salmonella, Neisseria, Brucella, Escherichia, Listeria, Francisella, Legionella, Yersinia, Staphylococcus, Clostridium, Shigella, or Streptococcus species.

In some embodiments, the Mycobacterium species is M. tuberculosis, M. leprae, M. lepromatosis, M. avium, M. kansasii, M. fortuitum, M. chelonae, M. marinum, or M. abscessus; the Salmonella species S. enterica, S. typhimurium, or S. bongori; the Neisserie species is N. gonorrhoeae or N. meningitidis; the Brucella species is B. melitensis, B. abortus, B. suis, or B. canis; the Escherichia species is E. coli; the Listeria species is L. monocytogenes; the Francisella species is F. tularensis, F. novicida, or F. philomiragia; the Legionella species L. pneumophila; the Yersinia species is Y. pestis or Y. enterocolitica; the Staphylococcus species is S. aureus; the Clostridium species is C. botulinum, C. perfringens, C. tetani, or C. sordellii; the Shigella species is S. dysenteriae, S. flexneri, S. boydii, or S. sonnei; or the Streptococcus species is S. pyogenes, S. agalactiae, S. dysgalactiae, S. bovis, S. anginosus, S. sanguinis, S. mitis, S. mutans, or S. pneumoniae.

In some embodiments, the supramolecular structure may have a polydispersity index (PDI) of from about 0.05 to about 0.3. In some embodiments, the supramolecular structure may further include one or more lipids, e.g., an ionizable lipid. In some embodiments, the supramolecular structure may further include at least one targeting moiety.

In some embodiments, the targeting moiety is an extracellular targeting moiety targeting a professional antigen presenting cell (e.g., a macrophage or a dendritic cell).

In some embodiments, the targeting moiety includes phosphatidylserine.

In some embodiments, the targeting moiety includes an antibody or antigen-binding fragment thereof. The antibody or antigen-binding fragment therefore may be selected from the group consisting of anti-CD163, anti-CD40, anti-CD74, anti-CD206, anti-CD123 antibodies, and antigen-binding fragments thereof. The antibody or antigen-binding fragment thereof may be selected from the group consisting of anti-DEC205, anti-CD304, anti-CD303, anti-CD40, anti-CD74, anti-BDCA2, and anti-CD123 antibodies, and antigen-binding fragments thereof.

In some embodiments, the targeting moiety includes a pathogen-associated molecular pattern (RAMP).

In some embodiments, the targeting moiety is a mannose cluster or folate.

In some embodiments, the targeting moiety is a TLR2 agonist. For example, the TLR2 agonist may be selected from the group consisting of MALP-2 lipoprotein, MALP-404 lipoprotein, outer surface lipoprotein A (OspA), a porin, LcrV, Hsp60, glycoprotein gH/gL, or glycoprotein gB.

In some embodiments, the supramolecular structure is a lipid nanoparticle. In some embodiments, the supramolecular structure is a micelle. In some embodiments, the supramolecular structure is a liposome. The liposome may be unilamellar or multilamellar (e.g., 2, 3, 4, 5, or more lamellae). The supramolecular structure may have polydispersity index of from about 0.05 to about 0.3.

The supramolecular structure may further include one or more lipids (e.g., an ionizable lipid).

In some embodiments, the method further includes an antibiotic. In some embodiments, the antibiotic is selected from the group consisting of cephalosporins, carbapenems, penicillins, and fluoroquinolones. In some embodiments, the antibiotic is selected from the group consisting of thiacetazone, sq-109, bedaquiline, delamanid, pyrazinamide, and isoniazid. For example, the antibiotic may be azithromycin, clarithromycin, ethambutol, rifampin, or amikacin.

In some embodiments, the bacteriophage is capable of infecting the bacterial cell (e.g., mycobacterial cell, e.g., NTM cell). The bacteriophage may be a mycobacteriophage. The bacteriophage may include a polynucleotide encoding a lytic protein (e.g., a lysin, an amylase, or a capsule depolymerase). The lysin may be, e.g., Lysin A or Lysin B. The amylase may be, e.g., isoamylase or α-amylase.

In some embodiments, the composition is administered intravenously, orally, topically, or via inhalation.

In another aspect, the invention provides a method of isolating a phage targeted to a bacterium, the method including the steps of:

contacting a heterogeneous mixture that includes the phage and a detergent, a polar, water-immiscible, aprotic solvent is chloroform, or a combination thereof to produce a composition that includes a liquid and a solid;

separating the liquid from the solid to produce a supernatant;

concentrating the supernatant to produce an enriched supernatant;

incubating the enriched supernatant with the bacterium to produce a cell mixture that includes the phage, cells, and debris; and

separating the phage from the cells and debris to isolate the phage.

In some embodiments, the heterogeneous mixture is a sewage sludge. In some embodiments, the detergent is t-octylphenoxypolyethoxyethanol (TRITON X-100), polysorbate (e.g., TWEEN 20), or nonoxynol 9. In some embodiments, the polar, water-immiscible, aprotic solvent is chloroform.

### Definitions

As used herein, the term “about” refers to +/−10% of a recited value.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disease, is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be by any appropriate route including, but not limited to, oral routes, intravenous routes, intramuscular routes, topical routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “effective amount,” “therapeutically effective amount,” and “a “sufficient amount” of an agent that results in a therapeutic effect (e.g., in a cell or a subject) described herein refer to a quantity sufficient to, when administered to the cell or subject, including a human, effect beneficial or desired results, including pre-clinical or clinical results, and, as such, an “effective amount” or synonym thereto depends on the context in which it is being applied. For example, in the context of treating a disorder, it is an amount of the agent that is sufficient to achieve a treatment response as compared to the response obtained without administration. The amount of a given agent will vary depending upon various factors, such as the given agent, the pharmaceutical formulation, the route of administration, the severity of the mycobacterial infection, the identity of the subject (e.g., age, sex, and/or weight) or host cell (e.g., mammalian immune cell) being treated, and the like, but can nevertheless be routinely determined by one of skill in the art. Also, as used herein, a “therapeutically effective amount” of an agent is an amount which results in a beneficial or desired result in a cell or subject as compared to a control. As defined herein, a therapeutically effective amount of an agent may be readily determined by one of ordinary skill by routine methods known in the art. Dosage regimen may be adjusted to provide the optimum therapeutic response.

The term “endosomal escape moiety,” as used herein, represents a moiety which enhances the release of endosomal contents or facilitates for the escape of a molecule from an internal cellular compartment (e.g., an endosome, a phagosome, or a lysosome), as compared to a reference molecule that differs only in that it lacks an endosomal escape moiety.

As used herein, “lipid nanoparticle” or “LNP” is a vesicle that includes a lipid layer encapsulating a substantially solid lipid core; the lipid core can contain a pharmaceutically active molecule. LNPs typically contain a cationic lipid, a non-cationic lipid, and a lipid that prevents aggregation of the particle (e.g., a PEG-lipid conjugate).

As used herein, the term “liposome” refers to a vesicle composed of amphiphilic lipids arranged in at least one bilayer, e.g., one bilayer or a plurality of bilayers. Liposomes include unilamellar and multilamellar (e.g., 2, 3, 4, 5, or more lamella) vesicles that have a membrane formed from a lipophilic material and an aqueous interior. The aqueous portion contains the phage or mixture of phage and other components. The lipophilic material isolates the aqueous interior from an aqueous exterior, which typically does not include the phage protein, although in some examples, it may. Liposomes also include “sterically stabilized” liposomes, a term which, as used herein, refers to liposomes that include one or more specialized lipids that, when incorporated into liposomes, result in enhanced circulation lifetimes relative to liposomes lacking such specialized lipids.

“Micelles” are defined herein as a type of a supramolecular structure in which amphipathic molecules (e.g., lipids) collectively define a volume, e.g., a substantially spherical volume. Amphipathic molecules (e.g., lipids) typically make up a shell of a micelle. In this shell, the hydrophobic portions of the amphipathic molecules are typically directed inward, leaving the hydrophilic portions in contact with the surrounding aqueous phase. The converse arrangement exists if the surrounding medium is hydrophobic.

The term “targeting moiety,” as used herein, represents a moiety (e.g., a small molecule, e.g., a carbohydrate) that specifically binds or reactively associates or complexes with a receptor or other receptive moiety associated with a given target cell population (e.g., a professional antigen-presenting cell (e.g., macrophage or dendritic cell)). Thus, a targeting moiety may be used to target a supramolecular structure described herein to, e.g., a professional antigen-presenting cell (e.g., macrophage or dendritic cell).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the term “supramolecular structure” refers to a complex of molecules held together by noncovalent bonds, such as hydrogen bonds, Van der Waals forces, electrostatic interactions, hydrophobic effect, and Pi-Pi interactions. Supramolecular structures may include large complexes of molecules that form, e.g., sphere-like structures. Supramolecular structures include, for example, lipid-based supramolecular structures, such as liposomes and lipid nanoparticles (e.g., micelles).

As used herein, the term “targeted intracellular compartment” refers to an endosome, phagosome, lysosome, or cytosol.

The term “targeting moiety,” as used herein, represents a moiety (e.g., a small molecule, e.g., a carbohydrate) that specifically binds or reactively associates or complexes with a receptor or other receptive moiety associated with a given target cell population (e.g., a professional antigen-presenting cell (e.g., macrophage or dendritic cell)). Thus, a targeting moiety may be used to target a supramolecular structure described herein to, e.g., a professional antigen-presenting cell (e.g., macrophage or dendritic cell).

“Vesicles” are defined herein as a type of a supramolecular structure in which amphipathic molecules (e.g., lipids) collectively define a volume, e.g., a substantially spherical volume. Amphipathic molecules (e.g., lipids) typically make up at least one shell of a vesicle. In this shell, the amphipathic molecules are arranged in a bilayer with hydrophilic portions of the amphipathic molecules being outwardly directed relative to the plane of the bilayer and the hydrophobic portions of the amphipathic molecules being disposed predominantly within the bilayer. The converse arrangement exists if the surrounding medium is hydrophobic.

### BRIEF DESCRIPTION OF THE DRAWINGS

FIG. 1 is a schematic drawing of an experimental design to screen and quantitate the level of enzybiotic reactions in grown out serial dilutions (GOSD) of cells, including Mycobacterium cells or macrophages infected with Mycobacterium and treated with antibacterial lytic proteins or bacteriophages with therapeutic payloads, respectively. For example, the inset image depicts a GOSD plate of ECL55 cells infected with Mycobacterium and treated with antibacterial lytic proteins (ABIα) or dilution buffer (DB).

FIGS. 2A and 2B are a set of graphs showing the dose-dependent response of GOSD −1, −2, −3, −4, −5, −6, −7, and −8 of M. abscessus cells to the treatment of one (10 μL), two (20 μL), or three (30 μL) doses of the enzyme cocktail of Lysin A (A), Lysin B (B), Isoamylase (I), and α-amylase (α)(ABIα), respectively. FIG. 2A is a quantification of the dose-dependent effect of ABIα treatment on the culture optical density (OD590)/lower limit of detection (LLoD), while FIG. 2B is a quantification of effect of treatment on the number of M. abscessus cells.

FIG. 3 is a graph showing the number of M. abscessus cells following treatment with 3.2 μg, 1.6 μg, 0.8 μg, 0.4 μg, 0.2 μg, and 0.1 μg of ABIα/well or no ABIα, respectively.

FIGS. 4A and 4B are a graph (FIG. 4A) and representative image (FIG. 4B), depicting the OD590/LLoD of GOSD (−1, −2, −3, −4, −5, −6, −7, and −8) of M. intracellulare cells treated with or without one dose of ABIα and cultured for 3 or 6 days, respectively.

FIG. 5 is a tabular set of data depicting the Fractional Inhibitory Concentration index (FICI) of the growth of M. abscessus cells treated with ABIα and antibiotic amikacin, biapenem, cefoxitin, ethambutol, moxifloxacin, rifampicin, or clarithromycin, respectively.

FIG. 6 is a schematic drawing of an experimental design to evaluate the differential effect on attenuating mycobacterial growth in infected macrophages by treatment with unencapsulated (Free Enzymes) or encapsulated antibacterial lytic proteins (Encapsulated Enzymes).

FIG. 7 is a graph depicting the OD590/LLoD of GOSD (-1, −2, −3, −4, −5, −6, −7, and −8) of M. abscessus following their extraction from infected macrophages treated with combinations of unencapsulated (free) or encapsulated (enc) A, B, I, and a (ABIα, AB, Iα, Biα, encABIα, encAB, encIα, and encBiα), respectively.

FIG. 8 is table showing quantification of the number of M. abscessus cells in the experiment as described in FIG. 7.

FIG. 9 is a schematic drawing of an experimental design to evaluate the differential effect of attenuating mycobacterial growth in infected macrophages treated with unencapsulated (Free bacteriophage) or encapsulated bacteriophage.

FIG. 10 is a graph depicting the OD590/LLoD of GOSD (−1, −2, −3, −4, −5, and −6) of S. flexneri following their extraction from infected macrophages treated with unencapsulated (Phage) or encapsulated (encPhage) Shigella phage EPH34, respectively.

### DETAILED DESCRIPTION

Bacteriophages are viruses that infect and replicate within bacteria. Lytic phages infect a host bacterium, utilize the host machinery to replicate the virion. Following replication, the phage lyses the host cell, releasing phage progeny to find new bacterial hosts to infect. Bacteriophages are programmed with all the essential machinery to infect a host and utilize host machinery to propagate. Thus, bacteriophages represent attractive anti-bacterial therapies due to their ability to specifically target, infect, and destroy a bacterial host cell.

Several bacterial pathogens that reside within a host cell are challenging to target. These intracellular bacteria reside and multiply within host cells in order to evade immune detection. To effectively target intracellular bacteria, the therapeutic payload must be targeted not only to the bacteria, but also the correct subcellular location in which the bacteria resides. The present invention solves this problem by providing compositions and methods of use thereof for targeting anti-bacterial therapies to treat intracellular bacterial infections. In general, the compositions feature a supramolecular structure (e.g., lipid-based supramolecular structure, e.g., a liposome, micelle, lipid nanoparticle (LNP)) that targets the host cell (e.g., macrophage or dendritic cell) and the correct targeted intracellular compartment (endosome, phagosome, lysosome, or cytosol). Meanwhile, the supramolecular structure is pre-loaded with a bacteriophage primed to infect and kill the bacterial cell. The supramolecular structure directs the payload to the correct cell and intracellular compartment, while the bacteriophage directly targets the bacteria due to its unique surface recognition properties.

Bacteriophages loaded into supramolecular complexes may be administered to a subject. The supramolecular complex is endocytosed by a cell (e.g., a professional antigen presenting cell such as a macrophage or dendritic cell), and the bacteriophage is delivered to the targeted intracellular compartment (endosome, phagosome, lysosome, or cytosol) wherein the bacteria reside. The phage recognizes a bacterium within the targeted intracellular compartment and attaches to the bacterial surface. The phage genome, which encodes one or more lytic enzymes, is transcribed and translated by the host machinery. These lytic enzymes then break down the cell wall of the bacterium, leading to osmotic rupture and release of phage progeny that can infect and kill nearby bacteria cells. In mycobacteriophages, the lytic enzymes include lysins (e.g., Lysin A and Lysin B) and capsule depolymerases due to the tough barrier of the mycoenvelope. Capsule depolymerases are enzymes that break down the outer capsule of mycobacterial cells. The outer capsule is generally composed of polysaccharides and proteins, with a minor amount of lipids. Capsule polysaccharides in the capsule include, for example, D-glucan, D-arabino-D-mannan, and D-mannan. Capsule depolymerases are enzymes that break down polysaccharides containing these, and other polysaccharides found in the capsule. These enzymes are responsible for cleaving different layers of the mycoenvelope. For example, in some Mycobacteria, Lysin A cleaves the peptidoglycan layer, and Lysin B cleaves between the arabinogalatan and mycolate outer membrane. Some mycobacteriophages also encode holins, which are membrane proteins that oligomerize and permeabilize the membrane forming holes to allow lysins to access their substrate and disrupt the cell wall.

### Bacteriophages

Bacteriophages contain the machinery necessary to recognize a target bacterium and lyse it as a result of phage infection. Bacteriophages perform a set of functions that include gaining access to the host cell surface that contains surface receptors, docking on to the surface via a positive macromolecular interaction, injecting the genome into the target cell, expressing the viral genome into RNAs and proteins, assembling new viral particles with replicated genomes, and lysing the host cell to release the progeny phage. Some estimates suggest over 10³⁰ phages exist, illustrative of the vast diversity that provides a trove of bacteriolytic agents for the development of therapeutic agents.

Mycobacteriophages (phages) are double-stranded DNA viruses that specifically infect Mycobacteria, ultimately culminating in the death of mycobacterial cells at the end of a lytic infection cycle. Mycobacteriophages have evolved to possess specific lysis systems including lipolytic enzymes dedicated to targeting and lysing particular types of bonds in specific layers of the highly hydrophobic mycobacterial cell well, which includes a covalently linked mycolyl-arabinogalatan-peptidoglycan (mAGP) complex as the core of the cell wall.

The bacteriophages described herein infect intracellular bacteria, such as an intracellular bacterium that resides in a professional antigen presenting cell (e.g., macrophage or denritic cell). In some embodiments, the bacteriophage is capable of infecting Mycobacterium, Salmonella, Neisseria, Brucella, Escherichia, Listeria, Francisella, Legionella, Yersinia, Staphylococcus, Clostridium, Shigella, or Streptococcus species. In some embodiments, the bacteriophage is a mycobacteriophage that is capable of infecting a Mycobacterium. Examples of mycobacterium species include, without limitation, M. tuberculosis, M. leprae, M. lepromatosis, M. avium, M. kansasii, M. fortuitum, M. chelonae, M. marinum, or M. abscessus. The mycobacteriphage may infect any of the foregoing Mycobacteria species. In some embodiments, the mycobacteriophage may inflect a plurality of the foregoing Mycobacteria.

The bacteriophage may infect S. enterica, S. typhimurium, or S. bongori. The bacteriophage may infect N. gonorrhoeae or N. meningitidis. The bacteriophage may infect B. melitensis, B. abortus, B. suis, or B. canis. The bacteriophage may infect E. coli. The bacteriophage may infect L. monocytogenes. The bacteriophage may infect F. tularensis, F. novicida, or F. philomiragia. The bacteriophage may infect L. pneumophila. The bacteriophage may infect Y. pestis or Y. enterocolitica. The bacteriophage may infect S. aureus. The bacteriophage may infect C. botulinum, C. perfringens, C. tetani, or C. sordellii. The bacteriophage may infect S. dysenteriae, S. flexneri, S. boydii, or S. sonnei. The bacteriophage may infect S. pyogenes, S. agalactiae, S. dysgalactiae, S. bovis, S. anginosus, S. sanguinis, S. mitis, S. mutans, or S. pneumoniae.

In some embodiments, the bacteriophage is a naturally occurring bacteriophage. In other embodiments, the bacteriophage is an engineered bacteriophage. In some embodiments, the compositions described herein include two or more (e.g., 3, 4, 5, 6, 7, 8, 9, 10, or more) distinct bacteriophages, e.g., to target a bacterial infection caused by more than one species or strains of bacteria.

In some embodiments, the bacteriophage is a bacteriophage including a nucleic acid encoding, e.g., a lytic enzyme (e.g., a lysin (e.g., Lysin A and/or Lysin B), an amylase (e.g., isoamylase and α-amylase), a holin, or a capsule depolymerase. Non-limiting examples of lytic enzymes are provided in Table 1.

TABLE 1

| Protein | Accession Number |
|---|---|
| Lysin A | AAD17596.1 |
| Lysin B | AAD17597.1 |
| Gp4 (holin) | NP_046822.1; O64200.1 |
| Gp5 (holin) | NP_046823.1 |
| holin | BBC44138.1 (putative); O64204.1 |
| Lysin A | BBC44137.1 (putative); O64203.1 |
| Lysin B/mycolylarabinogalactan | BBC44139.1 (putative); O64205.1 |
| esterase | |
| Gp3 (Lysin B) | O64199.1 |
| Gp4 (holin) | AAG48320.1 |
| Gp3 (Lysin B) | AAG48319.1 |
| α-amylase | A0A2S8DRT8 |
| isoamylase | P10342 |

A bacteriophage targeted to a bacterium may be isolated from a source, e.g., a heterogeneous mixture (e.g., sewage sludge) by: contacting a heterogeneous mixture including the phage with a detergent (e.g., t-octylphenoxypolyethoxyethanol (TRITON X-100), polysorbate (e.g., TWEEN 20), or nonoxynol 9), a polar, water-immiscible, aprotic solvent, or a combination thereof to produce a composition including a liquid and a solid; separating the liquid from the solid to produce a supernatant; concentrating the supernatant to produce an enriched supernatant; incubating the enriched supernatant with the bacterium to produce a cell mixture including the phage, cells, and debris; and separating the phage from the cells and debris to isolate the phage. Advantageously, this method allows for the isolation of viable bacteriophages while destroying other viral pathogens that may be found in sewage, e.g., SARS-CoV-2 virus.

In some embodiments, the detergent (e.g., t-octylphenoxypolyethoxyethanol (TRITON X-100), polysorbate (e.g., TWEEN 20), or nonoxynol 9) is at a concentration of about 0.007% to about 0.1% w/v (e.g., about 0.008% to about 0.1% w/v, about 0.009% to about 0.1% w/v, about 0.001% to about 0.1% w/v, about 0.010% to about 0.1% w/v, about 0.020% to about 0.1% w/v, about 0.030% to about 0.1% w/v, about 0.040% to about 0.1% w/v, about 0.050% to about 0.1% w/v, about 0.060% to about 0.1% w/v, about 0.070% to about 0.1% w/v, about 0.080% to about 0.1% w/v, or about 0.090% to about 0.1% w/v). In some embodiments, the polar, water-immiscible, aprotic solvent is chloroform.

### Intracellular Bacteria

Intracellular bacteria reside within a host cell where they reproduce and cause infection. Intracellular bacteria may reside within immune cells, such as professional antigen cells. Professional antigen presenting cells (APCs) include macrophages, B cells, and dendritic cells. APCs process and display antigens complexed with major histocompatibility complexes (MHCs) on their surfaces. T cells recognize these antigen presentation complexes using T cell receptors, a process that is critical for effective adaptive immune response. Certain bacteria evade this immune response by hiding within the immune cell.

The compositions and methods described herein may be used to target any intracellular bacteria, such as an intracellular bacterium that resides in a professional antigen presenting cell (e.g., macrophage or dendritic cell). In some embodiments, the bacterial cell is a Mycobacterium, Salmonella, Neisseria, Brucella, Escherichia, Listeria, Francisella, Legionella, Yersinia, Staphylococcus, Clostridium, Shigella, or Streptococcus species. Examples of Mycobacterium species include M. tuberculosis, M. leprae, M. lepromatosis, M. avium, M. kansasii, M. fortuitum, M. chelonae, M. marinum, or M. abscessus. In particular embodiments, the Mycobacterium is a NTM. In some embodiments, the NTM is M. avium or M. abscessus. In some embodiments, the infection is caused by a combination of NTM, such as M. avium and M. abscessus.

Other intracellular bacteria are known in the art. The Salmonella species may be, e.g., S. enterica, S. typhimurium, or S. bongori. The Neisseria species may be, e.g., N. gonorrhoeae or N. meningitidis E. coli. The Brucella species may be, e.g., B. melitensis, B. abortus, B. suis, or B. canis. The Escherichia species may be, e.g., E. coli. The Listeria species may be, e.g., L. monocytogenes. The Francisella species may be, e.g., F. tularensis, F. novicida, or F. philomiragia. The Legionella species may be, e.g., L. pneumophila. The Yersinia species may be, e.g., Y. pestis or Y. enterocolitica. The Staphylococcus species may be, e.g., S. aureus. The Clostridium species may be, e.g., C. botulinum, C. perfringens, C. tetani, or C. sordellii. The Shigella species may be, e.g., S. dysenteriae, S. flexneri, S. boydii, or S. sonnei. The Streptococcus species may be, e.g., S. pyogenes, S. agalactiae, S. dysgalactiae, S. bovis, S. anginosus, S. sanguinis, S. mitis, S. mutans, or S. pneumoniae.

### Supramolecular Structures

Supramolecular structures may be used to formulate an anti-bacterial agent (e.g., a bacteriophage, e.g., mycobacteriophage) for delivery. Supramolecular structures include a defined complex of molecules (e.g., lipids) held together by noncovalent bonds, such as hydrogen bonds, Van der Waals forces, electrostatic interactions, hydrophobic effect, and Pi-Pi interactions. Supramolecular structures may include large complexes of molecules that form sphere-, rod-, or sheet-like structures. Supramolecular structures include, for example, lipid-based supramolecular structures, such as micelles, liposomes, and LNPs. Supramolecular structures may have a predetermined size. The size of the structure may vary based on the components (e.g., size of phage) packed within the structure.

In some embodiments, a particular particle size is used to access a certain endocytic route to direct the structure to the appropriate intracellular compartment. The supramolecular structure may be endocytosed and delivered to the targeted intracellular compartment, e.g., via clathrin-dependent endocytosis or via caveolin-dependent endocytosis. The Z-average mean particle diameter of the structure may vary from, e.g., about 75 nm to about 750 nm (e.g., from about 250 nm to about 750 nm, or from about 75 nm to about 250 nm). Preferably, when the supramolecular structure is an LNP or micelle, the Z-average mean particle diameter is from about 75 nm to about 250 nm. Preferably, when the supramolecular structure is a vesicle (e.g., a liposome), the Z-average mean particle diameter is from about 250 nm to about 750 nm. Non-limiting examples of the Z-average mean particle diameters include, e.g., from about 75 nm to about 100 nm, e.g., from 75 nm to about 85 nm, e.g., about 80 nm, e.g., from about 80 nm to about 140 nm, from about 90 nm to about 130 nm, or from about 110 nm to about 130 nm, e.g., about 120 nm, e.g., from about 200 nm to about 300 nm, e.g., from about 250 nm to about 300 nm, from about 260 nm to about 290 nm, from about 260 nm to about 280 nm, from about 265 nm to about 275 nm, e.g., about 270 nm, e.g., from about 300 nm to about 400 nm, from about 400 nm to about 600 nm, e.g., from about 450 nm to about 550 nm, from about 475 nm to about 525 nm, from about 480 nm to about 520 nm, from about 490 nm to about 510 nm, from about 495 nm to about 505 nm, e.g., about 500 nm, e.g., about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, about 500 nm, about 505 nm, about 510 nm, about 515 nm, about 520 nm, about 525 nm, about 530 nm, about 535 nm, about 540 nm, about 545 nm, about 550 nm, about 555 nm, about 560 nm, about 565 nm, about 570 nm, about 575 nm, about 580 nm, about 585 nm, about 590 nm, about 595 nm, about 600 nm, about 605 nm, about 610 nm, about 615 nm, about 620 nm, about 625 nm, about 630 nm, about 635 nm, about 640 nm, about 645 nm, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, or about 750 nm. In particular embodiments, the supramolecular structure contains a Z-average mean particle diameter of about 75 nm to about 250 nm (e.g., about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm).

The mean particle diameter may be measured by zeta potential, dynamic light scattering (DLS), electrophoretic light scattering (ELS), static light scattering (SLS), molecular weight, electrophoretic mobility, size exclusion chromatography (SEC), field flow fractionation, or other methods known in the art. In particular embodiments, the mean particle diameter is measured by. In particular embodiments, the supramolecular structure contains a Z-average mean particle diameter of from about 75 nm to about 150 nm. In particular embodiments, the supramolecular structure contains a Z-average mean particle diameter of about 500 nm. In particular embodiments, the supramolecular structure contains a Z-average mean particle diameter of about 270 nm. In particular embodiments, the supramolecular structure contains a Z-average mean particle diameter of about 80 nm. One of skill in the art would appreciate that a population of supramolecular structures (e.g., liposomes, LNPs, or micelles) may have a range of Z-average mean particle diameters within the population. Thus, the population may be polydisperse. The population may have a polydispersity index of 0.3 or less (e.g., 0.05 to 0.3). The polydispersity index can be determined using DLS (see, e.g., ISO 22412:2017).

The supramolecular structures may be loaded with a predetermined number of phages or average number of phages per supramolecular structure. For example, the supramolecular structure may contain from about one phage to about 20 phages (e.g., 1 to 15, 1 to 10, 1 to 15, 1 to 3, 2 to 10, 2 to 5, 2 to 4, e.g., 1, 2, 3, 4, 5, 6, 7, 8, 9, 10, 11, 12, 13, 14, 15, 16, 17, 18, 19, or 20 phages). The number of phages per structure may depend on the size of the phage and the size of the structure.

The supramolecular structures may include an endosomal escape moiety. Supramolecular structures including an endosomal escape moiety may provide for an improved cytosolic delivery of the cargo (e.g., a therapeutic agent) included in the supramolecular structure. Endosomal escape moieties are known in the art. Preferably, an endosomal escape moiety is an ionizable lipid. Ionizable lipids are typically. The ionizable lipids may also serve as supramolecular structure-layer forming lipids. Non-limiting examples of ionizable lipids include those described in, e.g., WO 2019/067875; WO 2018/191750; and U.S. Pat. No. 9,999,671. Other exemplary endosomal escape moieties include fusogenic lipids (e.g., dioleoylphosphatidyl-ethanolamine (DOPE)); and polymers such as polyethylenimine (PEI); poly(beta-amino ester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for inclusion in, or conjugation to, to the supramolecular structures disclosed herein are provided, e.g., in WO 2015/188197; the disclosure of these endosomal escape moieties is incorporated by reference herein.

### Liposomes

Liposomes are useful for the transfer and delivery of phages ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the phage are delivered into the cell where the phage can specifically target and lyse a bacterial cell (e.g., mycobacterial cell, e.g., NTM cell) residing inside a mammalian immune cell. In some cases, the liposomes are also specifically targeted, e.g., to direct the phage to particular mammalian immune cell types and/or to particular intracellular compartments that typically harbor bacteria (e.g., mycobacteria) during infection (endosome, phagosome, lysosome, or cytosol). The composition of the liposome is usually a combination of phospholipids, usually in combination with steroids, such as cholesterol. Other phospholipids or other lipids may also be used. The physical characteristics of liposomes depend on pH, ionic strength, and the presence of divalent cations.

Preferably, a liposome described herein includes a phospholipid, more preferably, a glycerophospholipid, e.g., a phosphatidylserine. A phosphatidylserine is a glycerol molecule having two hydroxyl groups substituted with fatty acid ester moieties and one hydroxyl group substituted with a phosphodiester moiety that is covalently bonded to serine side chain. A typical structure of a phosphatidylserine is RO—CH₂—CH(OR)—CH₂—OP(O)(OH)—OCH₂CH(COOH)NH₂, or a salt thereof, where each R is independently a fatty acid acyl. Additionally, or alternatively, a liposome described herein may include, e.g., a lysophospholipid, e.g., a lysophosphatidylserine. A lysophosphatidylserine is a phosphatidylserine missing one of its two fatty acid ester moieties. A typical structure of a lysophosphatidylserine is RO—CH₂—CH(OR)—CH₂—OP(O)(OH)—OCH₂CH(COOH)NH₂, or a salt thereof, where one R is a fatty acid acyl, and the other R is H. Thus, in certain preferred embodiments, a liposome described herein includes RO—CH₂—CH(OR)—CH₂—OP(O)(OH)—OCH₂CH(COOH)NH₂, or a salt thereof, where each R is H or a fatty acid acyl, provided that at least one R is a fatty acid acyl.

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Cationic liposomes possess the advantage of being able to fuse to the cell membrane. Non-limiting examples of cationic lipids include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylamminonium chloride (DOTAP), N—(I-(2,3-dioleyloxy)propyl)-N,N,N-trimethylamminonium chloride (DOTMA), N,N-dimethyl-2,3-dioleyloxy)propylamine (DODMA), 1,2-DiLinoleyloxy-N,N-dinnethylaminopropane (DLinDMA), 1,2-Dilinolenyloxy-N,N-dimethylaminopropane (DLenDMA), 1,2-Dilinoleylcarbamoyloxy-3-dinnethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dinnethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yeethylazanediyedidodecan-2-ol (Tech G1), or a mixture thereof. The cationic lipid can include, for example, from about 20 mol % to about 50 mol % or about 40 mol % of the total lipid present in the particle.

Non-cationic liposomes, although not able to fuse as efficiently with the plasma membrane, are taken up by macrophages in vivo and can be used to deliver phages to macrophages. Anionic liposome compositions generally are formed from dimyristoyl phosphatidylglycerol, while anionic fusogenic liposomes are formed primarily from dioleoyl phosphatidylethanolamine (DOPE). The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, 1,2-dioleoyl-sn-glycero-3-phospho-L-serine (sodium salt, DOPS), or a mixture thereof. The non-cationic lipid can be, for example, from about 5 mol % to about 90 mol %, about 10 mol %, or about 58 mol % if cholesterol is included, of the total lipid present in the particle. In some embodiments, an ionizable/non-cationic lipid can be a combination of lipids described above, e.g., a combination of lipids including DOPC, DOPS, Chol, and DOPE.

The conjugated lipid that inhibits aggregation of liposomal particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % of the total lipid present in the particle. In some embodiments, the liposome composition further includes cholesterol at, e.g., about 10 mol % to about 60 mol % or about 50 mol % of the total lipid present in the particle.

Another type of liposomal composition is formed from phosphatidylcholine (PC) such as, for example, soybean PC, and egg PC. Another type is formed from mixtures of phospholipid and/or phosphatidylcholine and/or cholesterol. Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Pub. No. 20060058255, the linking groups of which are herein incorporated by reference.

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid; peptidases (which can be substrate specific); and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate linkers are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

### Lipid Nanoparticles

Anti-bacterial agents of in the invention may be fully encapsulated in a lipid formulation, e.g., a lipid nanoparticle (LNP). LNPs are extremely useful for systemic applications, as they exhibit extended circulation lifetimes following intravenous (i.v.) injection and accumulate at distal sites (e.g., sites physically separated from the administration site). The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipids include DODAC, DDAB, DOTAP, DOTMA, DODMA, DLinDMA, DLenDMA, DLin-C-DAP, DLin-DAC, DLin-MA, DLinDAP, DLin-S-DMA, DLin-2-DMAP, DLin-TMA.Cl, DLin-TAP.Cl, 1 DLin-MPZ, DLinAP, DOAP, DLin-EG-DMA, (DLin-K-DMA or analogs thereof, ALN100, MC3, Tech G1, or a mixture thereof. The cationic lipid can include, for example, from about 20 mol % to about 50 mol % or about 40 mol % of the total lipid present in the particle.

The ionizable/non-cationic lipid can be an anionic lipid or a neutral lipid including, but not limited to, DSPC, DOPC, DOPS, DPPC, DOPG, DPPG, DOPE, POPC, POPE, DOPE-mal, DPPE, DMPE, DSPE, 16-O-monomethyl PE, 16-O-dimethyl PE, 18-1-trans PE, SOPE, cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % of the total lipid present in the particle.

In some embodiments, the LNP further includes cholesterol at, e.g., about 10 mol % to about 60 mol % or about 50 mol % of the total lipid present in the particle.

### Micelles

Micelles are a particular type of molecular assembly in which amphipathic molecules are arranged in a spherical structure such that all the hydrophobic portions of the molecules are directed inward, leaving the hydrophilic portions in contact with the surrounding aqueous phase. Micelles may be made of lipids. The micelle phase is caused by the packing behavior of single-tail lipids in a bilayer. The difficulty filling all the volume of the interior of a bilayer, while accommodating the area per head group forced on the molecule by the hydration of the lipid head group, leads to the formation of the micelle. This type of micelle is known as a normal-phase micelle (oil-in-water micelle). Inverse micelles have the head groups at the center with the tails extending out (water-in-oil micelle).

Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength. The process of forming micelles is known as micellization and forms part of the phase behavior of many lipids according to their polymorphism.

### Targeting Moieties

A supramolecular structure described herein may include, e.g., a targeting moiety. A targeting moiety may be used to direct the supramolecular structure to a particular cell-type (e.g., a professional antigen-presenting cell (e.g., macrophage or dendritic cell)).Certain lipids (e.g., phosphatidyl serine) may be used in the supramolecular structure (e.g., a vesicle) both as a supramolecular structure layer-forming lipid and as a targeting moiety. The targeting moiety may be, e.g., an antibody or an antigen-binding fragment or an engineered derivative thereof (e.g., Fcab or a fusion protein (e.g., scFv)). The targeting moiety may be, e.g., a polypeptide. Alternatively, the targeting moiety may be, e.g., a small molecule (e.g., mannose or folate) or a cluster of small molecules (e.g., a cluster of mannoses). A targeting moiety may be associated with a supramolecular structure covalently or non-covalently.

### Small Molecules

The targeting moiety may be a small molecule capable of complexing a receptor expressed on the surface of the targeted cell. Non-limiting examples of small molecules that may be used as targeting moieties in the supramolecular structures described herein are phosphatidylserine, lysophosphatidylserine folate, mannose, and mannose clusters.

Preferably, the targeting moiety is phosphatidylserine or lysophosphatidylserine. More preferably, the targeting moiety is phosphatidylserine. Phosphatidylserine and/or lysophosphatidylserine may be present as a supramolecular structure layer-forming lipid that is non-covalently bonded to the rest of the supramolecular structure.

Folate may be used as a targeting moiety. In the supramolecular structures described herein, folate may be of the following structure:

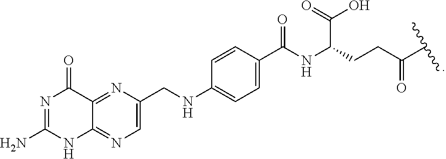 [SMILES: *C(=O)CC[C@H](NC(=O)c1ccc(NCc2cnc3[nH]c(N)nc(=O)c3n2)cc1)C(=O)O]

Mannose or a mannose cluster can be used to target the supramolecular structure described herein to dendritic cells and macrophages. Mannose clusters are known in the art.

Folate, mannose, and mannose clusters may be covalently linked to the supramolecular structure. Conjugation techniques for linking folate, mannose, and mannose clusters are known in the art, for example, as described in US 2014/0045919, U.S. Pat. Nos. 9,725,479, 8,758,810, 8,450,467, 6,525,031, 6,335,434, and5,759,572.

### Antigen-Binding Moieties

An antigen-binding moiety in the supramolecular structure described herein can be an antibody or an antigen-binding fragment thereof (e.g., F(ab)₂ or Fab) or an engineered derivative thereof (e.g., Fcab or a fusion protein (e.g., scFv)). A human or chimeric (e.g., humanized) antibody can be used as an antibody in the supramolecular structure described herein.

The antigen-binding moiety targets APCs having the surface antigen that is recognized by the antigen-binding moiety. Dendritic cells may be targeted by anti-DEC205, anti-CD304, anti-CD303, anti-CD40, anti-CD74, anti-BDCA2, or anti-CD123 antibodies or antigen-binding fragments thereof or engineered derivatives thereof. Macrophages can be targeted by anti-CD163, anti-CD40, anti-CD74, anti-CD206, or anti-CD123 antibodies or antigen-binding fragments thereof or engineered derivatives thereof.

Non-limiting examples of anti-CD38 antibodies are daratumumab, SAR650984, MOR202, or any one of antibodies Ab79, Ab19, Ab43, Ab72, and Ab110 disclosed in WO 2012/092616, the disclosure of these antibodies is incorporated herein by reference. A non-limiting example of an anti-CD79b antibody is huMA79b v28 disclosed in WO 2014/011521. A non-limiting example of an anti-CD22 antibody is 10F4 disclosed in US 2014/0127197. A non-limiting example of an anti-CD20 antibody is rituximab. A non-limiting example of an anti-DEC205 antibody is provided in US 2010/0098704, the antibodies of which are incorporated herein by reference. Non-limiting examples of anti-CD40 antibodies are lucatumumab and dacetuzumab. A non-limiting example of an anti-CD304 antibody is vesencumab.

Conjugation techniques for linking antigen-binding moieties are known in the art, for example, as described in Ansell et al., Methods Mol. Med., 25:51-68, 2000; US 2002/0025313; U.S. Pat. Nos. 6,379,699; and 5,059,421.

### Polypeptides

The targeting moiety can be a polypeptide having an affinity for cells (e.g., having an affinity for a cell type, e.g., a dendritic cell). Non-limiting examples of polypeptides are RGD peptide, rabies virus glycoprotein (RVG), and DC3 peptide. Alternatively, the polypeptide may be a TLR2 agonist, e.g., MALP-2 lipoprotein, MALP-404 lipoprotein, OspA, a porin, LcrV, Hsp60, glycoprotein gH/gL, or glycoprotein gB.

Conjugation techniques for linking peptides are known in the art, for example, as described in AnseII et al., Methods Mol. Med., 25:51-68, 2000; US 2002/0025313; U.S. Pat. Nos. 6,379,699; and 5,059,421.

### PAMPs

The targeting moiety may be a PAMP. PAMPs are known in the art, e.g., a CpG ODN. CpG ODNs are generally divided into three classes: class A, class B, and class C. Class A CpG ODNs typically contain poly-G tails with phosphorothioate backbones at the 3′- and 5′-termini and a central palindromic sequence including a phosphate backbone. Class A CpG ODNs typically contain CpG within the central palindromic sequence. Class B CpG ODNs typically include fully phosphorothioated backbone, and the sequence at the 5′ end of class B CpG ODNs is often critical for TLR9 activation. Class C CpG ODNs include a fully phosphorothioated backbone with a 3′-end sequence enabling formation of a duplex. A PAMP may be covalently linked to a supramolecular structure using techniques and methods known in the art.

### Methods of Treatment

The anti-bacterial agents described herein are preferably formulated into pharmaceutical compositions for administration to human subjects for the treatment of a disease or condition, such as a bacterial infection (e.g., intracellular bacterial infection, e.g., mycobacterial infection, e.g., NTM infection). Bacterial infections may occur in otherwise healthy subjects. Alternatively, the bacterial infection may occur in a subject with another comorbidity or disease. For example, a subject with a weakened immune system may be more susceptible to a bacterial infection.

Mycobacterial infections caused by NTM are bacteria that are normally present in the environment. Inhalation of these bacteria may cause disease in both healthy patients and those with compromised immune systems. NTM disease most often affects the lungs in adults, but it may also affect any body site. Some subjects are at higher risk of getting an NTM infection and developing disease. People who have an existing lung disease such as bronchiectasis (enlargement of airways), chronic obstructive pulmonary disease (COPD), cystic fibrosis, alpha-1 antitrypsin deficiency or who have had prior infections such as tuberculosis are at increased risk of pulmonary NTM disease. Subjects with advanced HIV infection (CD4<50) or immune-related genetic disorders (e.g., interferon-gamma deficiency or receptor deficiency, interleukin-12 deficiency) may develop pulmonary disease as part of a disseminated (e.g., widespread in the body) NTM infection. The subject to be treated may have any of the foregoing indications, e.g., in addition to a bacterial infection.

The methods compositions and methods described herein may be used to reduce a level of infection. For example, the methods may decrease a level of infection (e.g., number of bacteria or size of infection), as compared to a reference. For example, the infection may decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

### Pharmaceutical Compositions

The anti-bacterial agents described herein are preferably formulated into pharmaceutical compositions for administration to human subjects in a biologically compatible form suitable for administration in vivo.

The compositions described herein may be administered to a subject in a variety of forms depending on the selected route of administration, as will be understood by those skilled in the art. The compositions described herein may be administered, for example, by any route that allows the composition (e.g., supramolecular structure, e.g., liposome, micelle, or LNP) to reach the target cells. The composition may be administered, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. In one embodiment, the composition is administered via aero Parenteral administration may be by continuous infusion over a selected period of time. In some preferred embodiments, the compositions described herein are administered via inhalation.

Certain compositions described herein may be administered, e.g., by inhalation. Inhalation may be oral inhalation or nasal inhalation. An inhalable composition described herein may be provided as a liquid dosage form or dry powder dosage form. A dry powder composition may be, e.g., administered by inhalation as is or after reconstitution in a vehicle (e.g., saline (e.g., isotonic saline), phosphate-buffered saline, or water).

Inhalable dry powder dosage forms may be prepared from liquid compositions described herein by drying (e.g., by freeze drying, spray drying, spray-freeze drying, or supercritical fluid technology). Inhalable dry powder dosage forms described herein may include a carrier (e.g., lactose, sucrose, mannitol, and the like), cryoprotectant (e.g., trehalose, mannitol, and the like), and/or antiadherent (e.g., glycine, L-leucine, serine, and the like). Inhalable dry powder dosage forms described herein may be administered using dry powder inhalers. Dry powder inhalers are known in the art and may or may not include a propellant. Non-limiting examples of dry powder inhalers can be found in Newman, Expert Opin. Biol. Ther., 4:23-33, 2004, the disclosure of which is incorporated herein by reference in its entirety.

Inhalable liquid dosage forms (e.g., aerosol formulations) described herein may be prepared using techniques and methods useful in the preparation of liquid compositions containing supramolecular structures. Inhalable liquid dosage forms typically include a suspension of the supramolecular structures described herein in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form contains an aerosol dispenser, it will contain a propellant, which can be a compressed gas, e.g., compressed air or an organic propellant, e.g., hydrofluoroalkane. The inhalable liquid dosage forms may be administered using a nebulizer. The process of pneumatically converting a bulk liquid into small droplets is called atomization. The operation of a pneumatic nebulizer requires a propellant as the driving force for liquid atomization. Various types of nebulizers are described in Respiratory Care, 45:609-622, 2000, the disclosure of which is incorporated herein by reference in its entirety. Alternatively, an inhalable liquid dosage form described herein may be administered using a metered-dose inhaler. Metered-dose inhalers are known in the art and typically include a canister, actuator, and a metering valve.

A composition described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a composition described herein may be incorporated with an excipient and used in the form of ingestible tablets, buccal tablets, troches, capsules, elixirs, suspensions, syrups, and wafers. A composition described herein may also be administered parenterally. Solutions of a composition described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions suitable for buccal or sublingual administration include tablets, lozenges, and pastilles, where the active ingredient is formulated with a carrier, such as sugar, acacia, tragacanth, gelatin, and glycerin. Compositions for rectal administration are conveniently in the form of suppositories containing a conventional suppository base, such as cocoa butter.

The composition described herein may be administered to an animal, e.g., a human, alone or in combination with pharmaceutically acceptable carriers, as noted herein, the proportion of which is determined by the solubility and chemical nature of the composition, chosen route of administration, and standard pharmaceutical practice.

The dosage of the compositions (e.g., a composition including a bacteriophage) described herein, can vary depending on many factors, such as the pharmacodynamic properties of the phage, the mode of administration, the age, health, and weight of the recipient, the nature and extent of the symptoms, the frequency of the treatment, and the type of concurrent treatment, if any, and the clearance rate of the composition in the animal to be treated. The compositions described herein may be administered initially in a suitable dosage that may be adjusted as required, depending on the clinical response. In some embodiments, the dosage of a composition (e.g., a composition including a bacteriophage) is a prophylactically or a therapeutically effective amount. Furthermore, it is understood that all dosages may be continuously given or divided into dosages given per a given time frame. The composition can be administered, for example, every hour, day, week, month, or year. In some embodiments, the composition may be administered continuously or systemically.

### Combination Therapies

The pharmaceutical compositions described herein may be administered as part of a combination therapy. A combination therapy means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. Sequential or substantially simultaneous administration of each therapeutic agent can be by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection or via aerosolization while a second therapeutic agent of the combination may be administered, e.g., orally or via inhalation.

In any of the combination embodiments described herein, the first and second therapeutic agents may be administered simultaneously or sequentially, in either order. The first therapeutic agent may be administered immediately, up to 15 minutes, up to 30 minutes, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

The pharmaceutical compositions described herein may further include an additional anti-bacterial agent that is administered in conjunction with the supramolecular structure that includes a bacteriophage.

The compositions and methods described herein may further include treatment for an underlying lung condition, e.g., that may be exacerbated by a bacterial infection (e.g., NTM infection). Suitable lung therapies include, without limitation, airway clearance, nebulizers, respirators, inhalers (e.g., steroid inhalers).

### Antibiotics

The additional antibacterial agent may be an antibiotic. Suitable antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, chlorhexidine, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, trimethoprim, ethambutol, and rifampin. In some embodiments, multiple antibiotics are administered in combination with the compositions described herein. In some embodiments, the antibiotic is selected from the group consisting of cephalosporins, carbapenems, penicillins, and fluoroquinolones. In some embodiments, the antibiotic is selected from the group consisting of thiacetazone, sq-109, bedaquiline, delamanid, pyrazinamide, and isoniazid.

Advantageously, in some embodiments, the synergy with the co-administered therapeutic agents may permit the antibiotic to be administered at a dose that would be subtherapeutic, if administered without the other therapeutic agents.

The antibiotic may be formulated with the supramolecular structure containing the bacteriophage. The antibiotic may be administered as a separate pharmaceutical composition. The antibiotic may be administered at a different time than the pharmaceutical composition containing the supramolecular structure with phage. In some preferred embodiments, the additional antibiotic is amikacin. The amikacin may be liposomal amikacin that is formulated, e.g., for inhalation.

The following examples are meant to illustrate the invention. They are not meant to limit the invention in any way.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1: Materials and Methods

Cloning

I. Cloning of Lysin A

The open reading frame of bacteriophage Halo LysA (gp 10 (accession #NC_001900) was synthesized by Genscript without a stop codon into the NdeI to XhoI site of a pet21 a plasmid.

### II. Cloning of Lysin B

The open reading frame of bD29 LysB (gp12 accession #NC_001900) was synthesized by Genscript with adaptors to clone via Gibson based homology cloning at position 5237 of a pet21 plasmid downstream of a T7 tag.

Preparation of Proteins

I. Expression

E. coli BI21 (de3) cells were transduced with the pET21a plasmid including therapeutic payloads and were cultured in Tryptic Soy Broth (TSB) media. All cultures are grown for approximately 3.5 hours at 37° C. or until an optical density (OD) 600 of 0.4 was reached. Grown out cultures were incubated on ice for 30 minutes, then induced with 40 mM IPTG. Cultures were incubated overnight at a reduced temperature for expression. After removing from the incubator, cultures were centrifuged for 20 minutes at 4,000 revolutions per minute (RPM) in a swing bucket rotor to separate cells from the supernatant.

To purify the therapeutic proteins, lysis was performed with a solution containing Bacterial Protein Extraction Reagent (B-PER), benzonasem and lysozyme. The fast protein liquid chromatography (FPLC) running buffer consisted of 50 mM Tris pH 8, 250 mM NaCl, 50 mM imidazole (pH 8), 0.5 mM MgCl₂, and 10% glycine (glyc). The elution buffer consisted of 50 mM Tris pH 8, 250 mM NaCl, 700 mM imidazole (pH 8), 0.5 mM MgCl₂, and 10% glycerol. 5 mL of Cytiva HisTrap FF 5 was used with AktaPurifier and dialysis of the elution fractions were performed with 10 kDa dialysis bags overnight at 4° C. The dialysis buffer consisted of 50 mM Tris pH 8, 250 mM NaCl, 0.5 mM MgCl2, and 20% glycerol.

II. Liposome Encapsulation of Payloads

To assemble the liposomes with a Nanoassemblr Ignite (Precision Nanosystems), 1 mg/mL total lipid concentration of 1,2-Dioleoyl-sn-glycero-3-phosphocholine (DOPC): 1,2-dioleoyl-sn-glycero-3-phospho-L-serine (sodium salt; DOPS): cholesterol (Chol): 1,2-dioleoyl-sn-glycero-3-phosphoethanolamine (DOPE) at a ratio of: 1:0.3:0.4:1 were combined with the organic solvent Ethanol and aqueous buffer 1 M Tris pH 7.4.

To mediate payload additions to the liposomes: 2-4 mg/mL of the protein payload were added to the aqueous buffer, followed by the addition of 1×10⁸-1×10¹⁰ pfu/mL phage to the aqueous buffer. This occurred at a15 mL/min total flow rate and a 1:1 flow rate ratio; and with 1 mL total volume. To purify the liposomes by two step dialysis, dialysis through a 10 kDa slide-a-lyzer for 30 min was performed at room temperature in 100 mM or 10 mM Tris pH 7.4 5% glycerol. The sizes of samples were analyzed on with a dynamic light scattering (DLS) device to determine the size and polydispersity of encapsulated samples. Samples producing sizes between 0.5 and 1.5 microns were chosen for assay on cells.

Thus, ABIα (lysin A, lysin B, isoamylase, and α-amylase) and BIα (lysin B, isoamylase, and α-amylase) were prepared.

Killing Non-Tuberculosis Mycobacteria with Dosing of ABIα

On day 0, a suspension of M. abscessus bacterial cells was prepared by adjusting the logarithmic growing of cells to 0.5 by the McFarland standard in Middlebrook 7H9 broth+TWEEN (˜1×10⁸ cfu/mL) and diluting the adjusted suspension at a ratio of 1:5 in 7H9 broth+TWEEN. The media solution for the assay included 30 mL Middlebrook 7H9, ADC, and TWEEN; 3.3 mL 12× PM Additive (Biolog, Inc), 3.3 mL 1F-01a fluid (Biolog, Inc), and 400 μL of 100× Dye G (Biolog, Inc). The assay plate was prepared by pipetting 90 μL of the prepared media solution into the wells of a 96 well plate, adding 5 μL of the prepared cell suspension, and adding 10 μL of enzymes for a final concentration of 3.2 μg each. Controls included cells only with or without dialysis buffer. Following the preparatory steps, the assay plates were incubated by covering the plates with a lid and wrapping with parafilm. Plates were then placed in a 37° C. static incubator.

On Day 1, the dosing assay plate was prepared by adding 10 μL of enzyme cocktails to the wells designated as dose 2× and 3×. On Day 2, 10 μL of a ABIα (lysin A, lysin B, isoamylase, and α-amylase) or a BIα (lysin B, isoamylase, and α-amylase) cocktail were added to wells designated as Dose 3×. On Day 3, the dye media for serial dilutions was prepared with 50 mL of Middlebrook 7H9, ADC, and TWEEN and 500 μL of 100× Dye G. The grow out serial dilutions (GOSD e.g., −1, −2, −3, −4, −5, −6, −7, and −8) were prepared by aliquoting 90 μL of media to wells in 96 well plates, removing the assay plate from the incubator, pipetting 10 μL of reaction to the top row of a 96 well plate, and performing 10-fold serial dilutions from row A-H by pipetting 10 μL (e.g., pipette well to mix before dilution down to next row). Plates were then covered with a lid and wrapped with parafilm and placed in a 37° C., CO₂ incubator, static for 72 hours. Following incubation, the culture optical density (OD590) was read. The data of the experiments are presented as a histogram with the Y-axis depicting culture optical density (OD590) 590 divided by the lower limit of detection (LLoD)(OD590/LLoD). The LLoD was calculated with no cell controls for background determination, such that any value above 1 is significant for growth at that dilution.

Killing of Slow-growing Mycobacteria M. Intracellulare

Media solution was prepared with 5 mL of 12× PM Additive Solution (Biolog, Inc), 45 mL Middlebrook 7H9 (with ADC supplement), and 500 ul 100× Dye G (Biolog, Inc). The treatment of M. intracellulare was performed as follows: 90 μL of the prepared media solution was added to row A of a 96 well plate. The cell density of a growing strain of M. intracellulare was adjusted to 0.5 McFarland Standard (˜1×10⁸ cfu/mL) and 10 μL of cells were added to a 96 well plate to target input cell concentrations of 1×10⁵ cfu/well. As a control, some wells were left and/or 10 ul of dialysis buffer (DB) was added. 10 μL of ABIα cocktail was for final concentration of 3.2 μg/well and the plate was incubated at 37° C. for 3 days. As described before, the OD590 was read and the plate was placed back into the incubator. On day 6, the plate was incubated at 37° C. and OD590 was quantified. In a final step, a GOSD reaction was performed on cells alone, cells+DB, and cells+ABIα. To do so, 90 μL of media solution were added to a new 96 well plate, followed by the addition of 10 μL of reaction to the top row, and performing a 10-fold serial dilution from row A down to row H. The plate was incubated at 37° C. for 6 days and the OD590 was read.

MIC Determination of ABIα Cocktail on M. Abscessus

A tube of M. abscessus was diluted to the Macfarland standard 0.5, diluted to 1×10⁵ cells and added to wells according to a platemap. Then, 10 μL of ABIα cocktail (3.2 μg) were added to each well. In untreated controls the equivalent volume of DB was added. Next, Biapenem was added to wells in a 2-fold dilution series. All wells were brought to the appropriate volume and buffer condition with 1× dye media. Specifically, in a 50 mL falcon tube: 30 mL Middlebrook 7H9, ADC, and TWEEN; 3.3 mL 12× PM Additive, 3.3 mL IF-01 a fluid, and 400 ul 100× Dye G were added.

Plates were incubated at 37° C. with a lid and parafilm wrapped around sides for 24 hours. Before all serial dilutions, the static plates were shaken for at least 10 minutes and also pipetted up and down to break up aggregates. Replica GOSD experiments were performed for 96 hours at 37° C. in Omnilog Biolog, inc. In these experiments, dilutions were performed out to 1×10⁻⁶.

Uptake of Mycobacteria into Macrophages

On Day 1, macrophages were prepared as follows: a confluent culture of macrophages in a T-75 flask were decanted of media and replenished with fresh C-DMEM (10 mLs). Cells were scraped from bottom of flask with a cell scraper with the confluency at approximately 8×10⁶ cells and diluted in C-DMEM to a concentration of 1×10⁵cells/mL. 100 μL of diluted cells were added to the wells of a 96 well tissue culture plate (for a seeding density of 1×10⁴ cells/well) and the plate was incubated overnight at 37° C. in 5% CO₂.

On Day 2, the uptake of M. abscessus into macrophages was performed as follows: a growing culture of M. abscessus strain was adjusted to a 0.5 McFarland standard, diluted 1:5 in Middlebrook 7H9, and taken out the 96 well tissue culture plate and wash wells 3× with 100 μL phosphate buffered saline (PBS). 100 μL of fresh, prewarmed C-DMEM, 5 μL of 1×10⁵ M. abscessus cell z were added to wells. Plates were then incubated at 37° C. in 5% CO2 for three hours. After incubation, cells were washed three times with 100 μL of PBS. Next, 100 μL of C-DMEM with 250 μg/mL of Amikacin were added and the plate was incubated for 1 hour. Cells were washed three times with 100 μL of PBS and replenished with 85 μL of C-DMEM with 50 μg/mL Amikacin. To treat with enzymes, 15 μl of the prepared enzyme cocktails were added to designated wells of the tissue culture plate. Free enzyme treatment was performed with 3.2 μg of each component of the ABIα cocktail (e.g., lysin A only, lysin B only, isoamylase only, and a-amylase only). Similar quantities of encapsulated ABIα were added with an average of 50% encapsulation of the payload. Plates were incubated for 72 hours at 37° C. in 5% CO₂.

On Day 5, macrophage extraction was performed as follows: after incubation, media was removed, and cells were washed three times with PBS. 100 μL of 0.5% SDS was added and mixed by pipetting up and down. The plate was incubated at 37° C. for 10 minutes and removed from the incubator. Wells were pipetted up and down to mix and the media was transferred to new 96 well plate. The plate was added to a Biotek shaker incubator to disaggregate cells and GOSD was performed to all reaction wells for CFU quantification.

Shigella Phage Hunt Protocol and Phage Preparation

Shigella Phage Hunt Protocol and phage preparation were performed as follows. 500 mL of activated sludge sample from Deer Island Wastewater Treatment Facility were collected. Upon receipt, samples were treated TRITON X-100 to a final concentration of 0.1%. The bottle was inverted 5× and let to sit for 5 minutes. Samples were aliquoted into 50 mL falcon tubes and centrifuged for 10 minutes at 4000×g. The supernatants were collected into a clean bottle and treated once more with TRITON X 100 to a final concentration of 0.1%. For storage, 40% glycerol was added to supernatants to a final concentration of 20%. Supernatants were aliquoted in 40 mL volumes in 50 mL falcon tubes and stored in a −80° C. freezer until used.

40 mL of a TRITON-treated sewage supernatant that was stored in −80 in 20% glycerol was thawed. To a 250 mL flask, 50 mL of TSB and 1 mM magnesium chloride, and 500 μL of an overnight Shigella flexneri strain (ATCC 29903) were added. The sewage supernatant was concentrated with a Innovaprep Hollow Fiber pipette to provide enrichment. Next, the flask containing TSB and cells was eluted with one pump of elution fluid (0.075% TWEEN 20+25 mM Tris pH 8.0). The flask was incubated with shaking at 37° C. overnight. After incubation, the cells were spun down for 10 minutes at 4000×g. The supernatant was filtered through a 0.2 μm filter. To perform the plaque assay, 100 μL of cells were added to 100 μL of enrichment and serial dilutions of enrichment, mixed with 3 mLs of 0.5% TSB top agar, were plated onto TSA plates. The plates were incubated at 37° C. overnight. Afterwards, the plaques were grown up is 5 mL of TSB with 1 mM CaCl₂ for 24 hours. Cells and debris were pelleted @ 4000×g for 10 minutes and the supernatant was purified on Innovaprep, as before, and brought up in 1.5 mL phage buffer containing 50 mM Tris pH 8.0, 150 mM NaCl, 10 mM MgCl₂, 2 mM CaCl₂, and 0.1% gelatin.

Killing Intracellular Shigella with Shigella Phage

The Killing of Intracellular Shigella with Shigella phage was performed as follows. On Day 1 preparation of macrophages began with a confluent culture of macrophages in a T-75 flask which were decanted of media and replenished with fresh C-DMEM (10 mL). Cells were scraped from the bottom of the flask with a cell scraper (confluency=˜8×10⁶ cells) and diluted in C-DMEM to a concentration of 1×10⁵ cells/mL. 100 μL of diluted cells were added to wells of a 96 well tissue culture plate (for a seeding density of 1×10⁴ cells/well) and the plate was incubated overnight at 37° C. in 5% CO2. In order to make up liposomes for ABIα and Shigella experiments, a standard lipid makeup was used for all liposomes, including DOPC, DOPS, DOPE, and Chol. This makeup occurred with a 1:1 flow rate ratio and 15 mL/min flow rate. The lipid solvents used included EtOH and Aqueous buffer 1M Tris pH 8. All liposomes were dialyzed in 10 kDa membranes for 1 hour. Specifically, a 30-minute dialysis occurred in 100 mM Tris pH 8 5% glycerol and a 30-minute dialysis occurred in 10 mM Tris pH 8 5% glycerol. Measurement of the concentration of proteins occurred before adding cells by taking a sample, diluting it 1:1 in 50% IPA 50% PBS, vortexing, and reading the absorbance at 280 nm on a Nanodrop.

On Day 2 Uptake of bacteria into macrophages was performed by using a growing culture of Shigella flexneri, adjusting the culture to a 0.5 McFarland standard and confirming with a turbidometer, diluting 1:5 in Middlebrook 7H9 and taking out the 96 well tissue culture plate and washing the wells three times with 100 μL PBS. 100 μL of fresh, prewarmed C-DMEM and 5 μ of prepared cultures were added to the wells. The plates were incubated at 37° C. in 5% CO₂ for three hours. After incubation, cells were washed three times with 100 μL of PBS. 100 μL of C-DMEM with 250 μg/mL Amikacin were added and incubated for 1 hour. Afterwards, cells were washed with 100 μL of PBS and replenished with 85 μL of C-DMEM with 50 ug/mL Amikacin. Controls were left with no amikacin added. To post-treat with unencapsulated (e.g., free) or encapsulated payloads, 10 μL of the prepared payloads were added to designated wells of the tissue culture plate.

### Example 2: Delivery of Antibacterial Lytic Proteins Mediates Efficacious Killing of Mycobacterium Cells

This Example describes the demonstration of a dose-dependent enzymatic cocktail capable of attenuating reproduction of Mycobacterium cells.

Materials and Methods

Materials and Methods and cell lines are described in Example 1.

Results

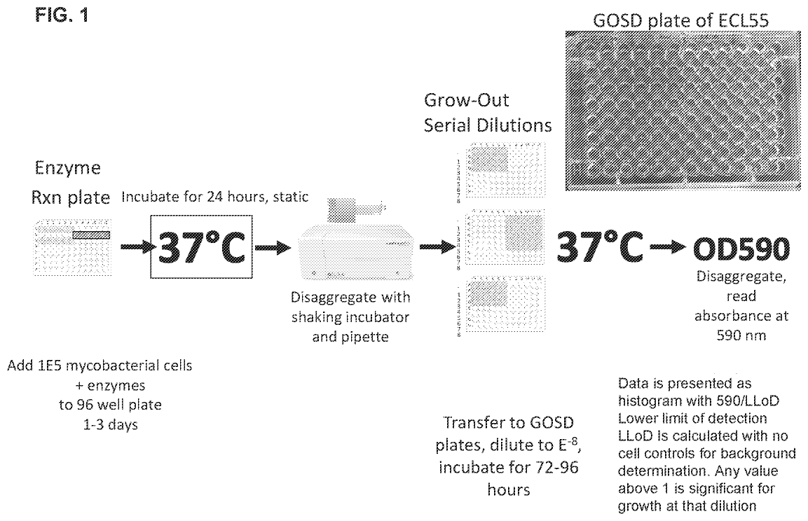

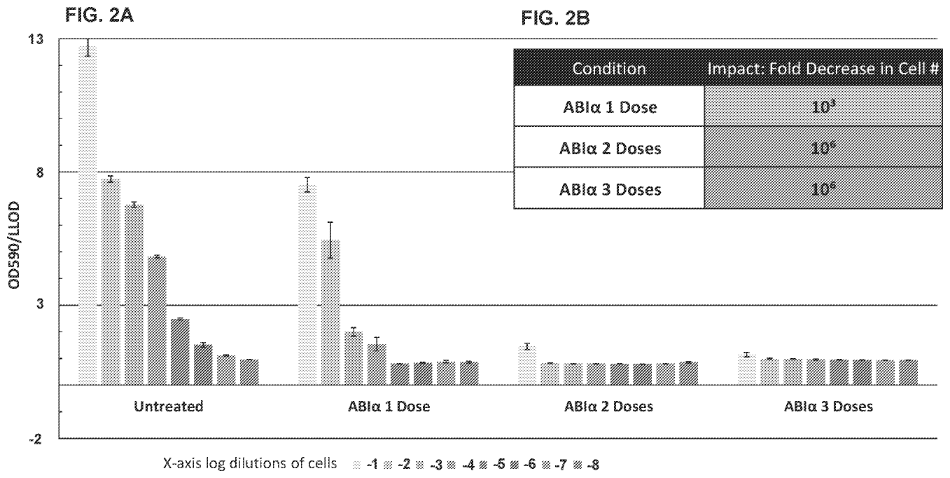

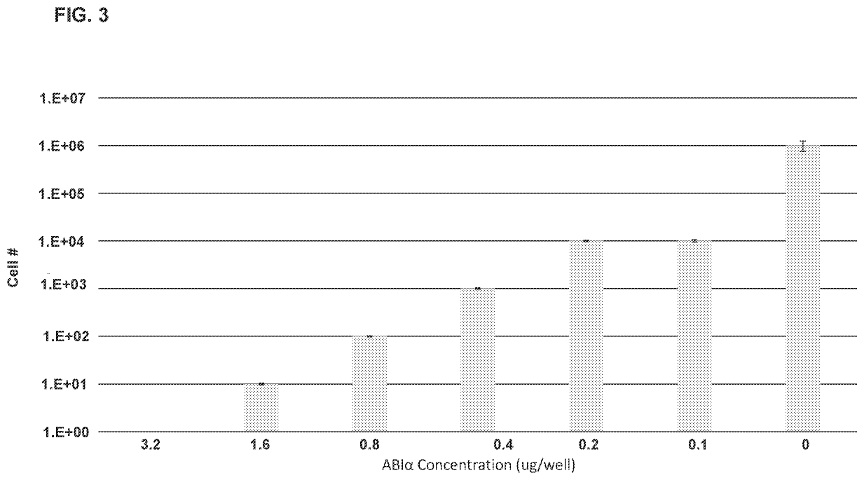

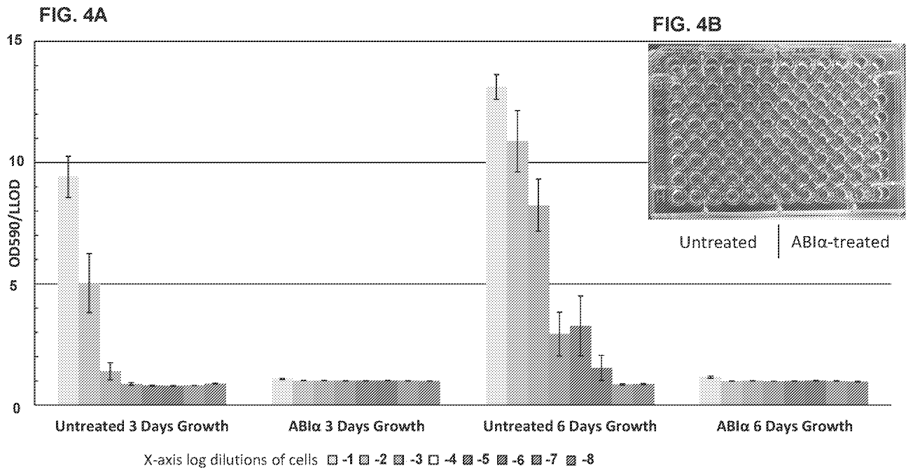

Screening of the growth of bacterial cells in a series of grown-out serial dilutions (GOSD) (FIG. 1) revealed that Mycobacterium abscessus (M. abscessus) were dose-dependently killed when treated with a single or multiple doses of the enzyme cocktail of Lysin A (A), Lysin B (B), Isoamylase (I), and α-amylase (α)(ABIα), as compared to untreated controls and as assessed by optical density (OD; FIG. 2A) and cell counting (FIG. 2B). To identify whether differential doses of ABIα treatment would elicit M. abscessus sterility, a dose-response experiment was conducted. FIG. 3 depicts the dose-response experiment, whereby it was observed that a 3.2 μg treatment of ABIα abolished M. abscessus. When similar experiments were conducted with the slow-growing Mycobacterium species M. intracellulare and cell growth was monitored (FIG. 4B) across several days, it was revealed that ABIα treatment led to the sustained attenuation of cell growth (FIG. 4A). In similar studies, it was demonstrated that the ABIα cocktail effectively attenuated the cell growth of M. avium, M. fortuitum, M. goodii, M. masiliense, M. boletti, M. chimera, and M. smegmatis (data not shown). Taken together, these results demonstrate the dose-dependent sterilization of mycobacteria by ABIα in an in vitro death/growth assay.

### Example 3: Delivery of Antibacterial Lytic Proteins and Antibiotics Mediates Synergistic Killing of Mycobacterium Cells

This Example describes the synergistic effect of the combination of an ABIα enzymatic cocktail and antibiotics on the attenuation of Mycobacterium reproduction.

Materials and Methods

Materials and Methods and cell lines are described in Example 1.

Results

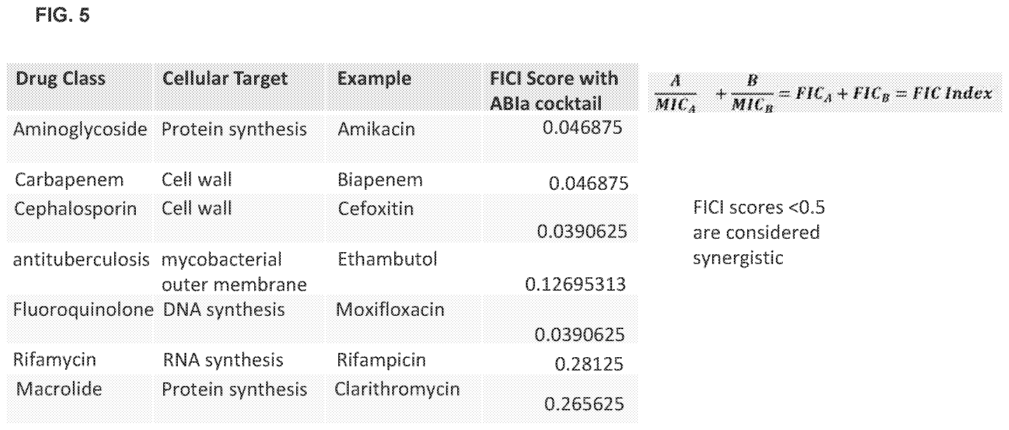

FIG. 5 is a tabular quantification of a cell growth assay of M. abscessus following treatment with ABIα and the antibiotic amikacin, biapenem, cefoxitin, ethambutol, moxifloxacin, rifampicin, or clarithromycin, respectively. Using the Fractional Inhibitory Concentration (FIC) index for quantitation revealed that ABIα in combination with amikacin, biapenem, cefoxitin, or moxifloxacin elicited synergistic effects on the attenuation of M. abscessus growth. Taken together, these results demonstrate that the enzymatic cocktail of ABIα in combination with a variety of chemical antibiotics elicits a synergistic effect on the death of Mycobacterium cells.

### Example 4: Delivery of Encapsulated Antibacterial Lytic Proteins Mediate Highly Efficacious Killing of Mycobacterium in Infected Macrophages

This Example describes the enhanced efficacy of an encapsulated enzymatic cocktail in the attenuation of reproduction of intracellularly localized Mycobacterium in infected macrophages.

Materials and Methods

Materials and Methods and cell lines are described in Example 1.

Results

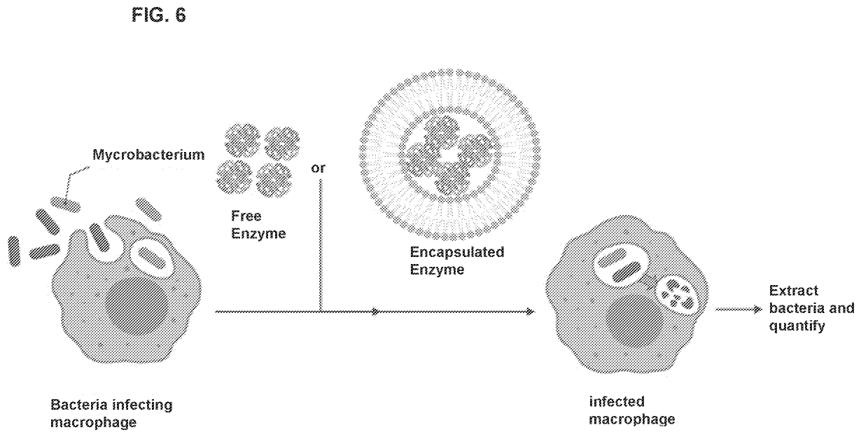

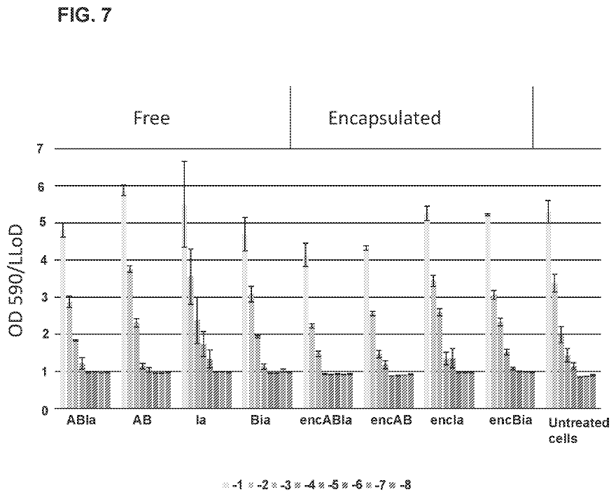

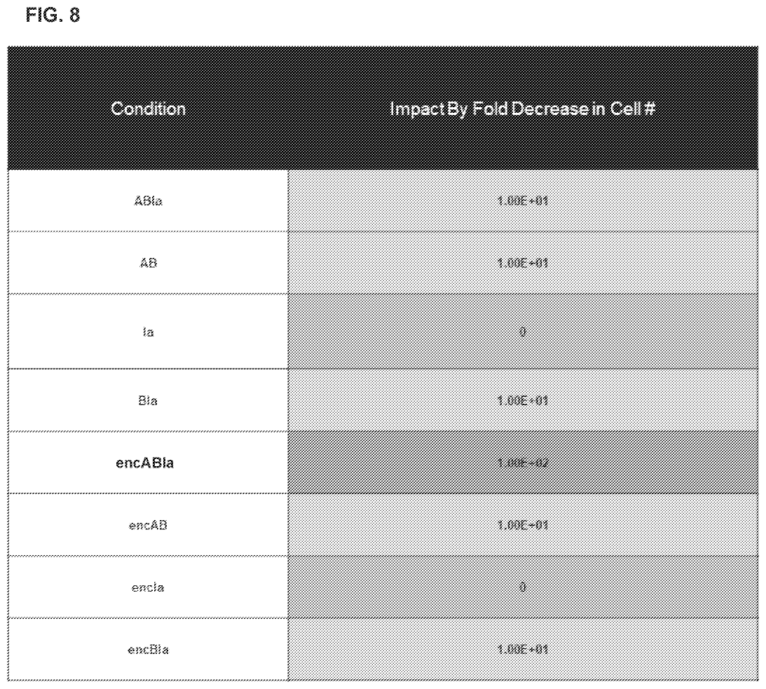

To identify the most efficacious combination of enzymes (lysin A (A), lysin B (B), isoamylase (I), or α-amylase (α)) and whether or not their encapsulation would enhance the efficacy of attenuating Mycobacterium reproduction (FIG. 6), the optical density of M. abscessus following their extraction from infected macrophages treated with unencapsulated ABIα, AB, Iα, or BIα; or encapsulated ABIα, AB, Iα, or BIα (encABIα, encAB, encIα, and encBIα, respectively; FIG. 7) was measured. It was observed that the encapsulated ABIα cocktail demonstrated enhanced killing of M. abscessus, as compared to unencapsulated ABIα and all other partial cocktails (FIG. 8). Taken together, these results demonstrate that a single dose of the encapsulated ABIα cocktail killed 99% of intracellular M. abscessus compared to 90% killing with unencapsulated ABIα.

### Example 5: Delivery of Encapsulated Bacteriophages Mediate Highly Efficacious Killing of Mycobacterium in Infected Macrophages

This Example describes the efficacy of an encapsulated Shigella bacteriophage in the attenuation of reproduction of intracellularly localized Shigella flexneri (S. flexneri) in infected macrophages.

Materials and Methods

Materials and Methods and cell lines are described in Example 1.

Results

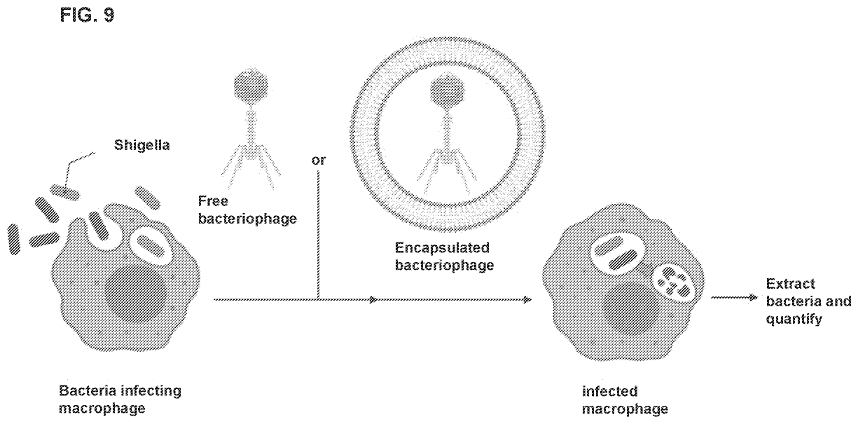

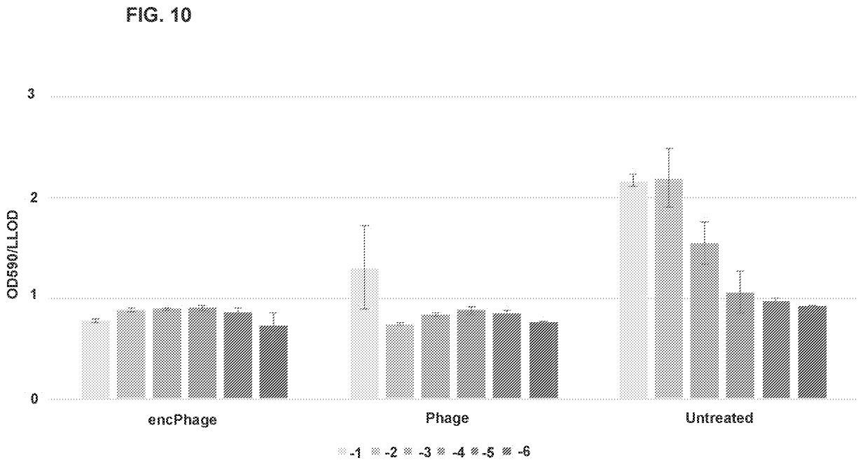

To identify whether Shigella bacteriophages could be used to attenuate the reproduction of S. flexneri in infected macrophages, an experiment was conducted in which macrophages were treated with S. flexneri and with unencapsulated or encapsulated Shigella phage encoding a therapeutic payload (EPH34) (FIG. 9). Following a 48-hour incubation period, cells were extracted and the GOSD were quantified overnight. It was observed that the encapsulated Shigella phage EPH34 (encPhage) displayed no growth after extraction from macrophages, while the unencapsulated phage EPH34 showed growth at the −1 dilution (FIG. 10). Taken together, these results demonstrated that the encPhage yielded the most efficacious reduction of S. flexneri in infected macrophages, as indicated by the 10-fold reduction of optical density.

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and that this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

1. A method of delivering a bacteriophage to a targeted intracellular compartment comprising a bacterial cell in a professional antigen presenting cell in a subject, the method comprising administering a composition comprising a supramolecular structure comprising the bacteriophage, wherein the supramolecular structure comprises a Z-average mean particle diameter of from about 75 nm to about 750 nm to the subject, wherein, following the administering step, the bacteriophage is delivered to the targeted intracellular compartment.

2. The method of claim 1, wherein the professional antigen presenting cell is a macrophage or a dendritic cell.

3. A method of treating an intracellular bacterial infection caused by a bacterial cell, the method comprising administering a composition comprising a supramolecular structure comprising a bacteriophage, wherein the supramolecular structure comprises a Z-average mean particle diameter of from about 75 nm to about 750 nm to the subject in an amount and for a duration sufficient to treat the bacterial infection.

4. The method of any one of claims 1 to 3, wherein the supramolecular structure comprises a Z-average mean particle diameter of from about 250 nm to about 750 nm.

5. The method of any one of claims 1 to 3, wherein the supramolecular structure comprises a Z-average mean particle diameter of from about 75 nm to about 250 nm.

6. The method of any one of claims 1 to 5, wherein the supramolecular structure further comprises a targeting moiety.

7. A method of delivering a bacteriophage to a targeted intracellular compartment comprising a bacterial cell in a professional antigen presenting cell in a subject, the method comprising administering a composition comprising a supramolecular structure comprising a targeting moiety and a cargo comprising the bacteriophage to the subject, wherein, following the administering step, the bacteriophage is delivered to the targeted intracellular compartment.

8. The method of claim 7, wherein the professional antigen presenting cell is a macrophage or a dendritic cell.

9. A method of treating an intracellular bacterial infection caused by a bacterial cell, the method comprising administering a composition comprising a supramolecular structure comprising a targeting moiety and a cargo comprising a bacteriophage to the subject in an amount and for a duration sufficient to treat the bacterial infection.

10. The method of any one of claims 7 to 9, wherein the supramolecular structure comprises a Z-average mean particle diameter of from about from about 250 nm to about 750 nm.

11. The method of any one of claims 7 to 9, wherein the supramolecular structure comprises a Z-average mean particle diameter of from about 75 nm to about 250 nm.

12. The method of any one of claims 1 to 11, wherein the bacterial cell is a Mycobacterium, Salmonella, Neisseria, Brucella, Escherichia, Listeria, Francisella, Legionella, Yersinia, Staphylococcus, Clostridium, Shigella, or Streptococcus species.

13. The method of claim 12, wherein:
(a) the Mycobacterium species is M. tuberculosis, M. leprae, M. lepromatosis, M. avium, M. kansasii, M. fortuitum, M. chelonae, M. marinum, or M. abscessus;
(b) the Salmonella species S. enterica, S. typhimurium, or S. bongori;
(c) the Neisserie species is N. gonorrhoeae or N. meningitidis;
(d) the Brucella species is B. melitensis, B. abortus, B. suis, or B. canis;
(e) the Escherichia species is E. coli;
(f) the Listeria species is L. monocytogenes;
(g) the Francisella species is F. tularensis, F. novicida, or F. philomiragia;
(h) the Legionella species L. pneumophila;
(i) the Yersinia species is Y. pestis or Y. enterocolitica;
(j) the Staphylococcus species is S. aureus;
(k) the Clostridium species is C. botulinum, C. perfringens, C. tetani, or C. sordellii,
(l) the Shigella species is S. dysenteriae, S. flexneri, S. boydii, or S. sonnei; or
(m) the Streptococcus species is S. pyogenes, S. agalactiae, S. dysgalactiae, S. bovis, S. anginosus, S. sanguinis, S. mitis, S. mutans, or S. pneumoniae.

14. The method of claim any one of claims 6 to 13, wherein the targeting moiety is an extracellular targeting moiety targeting a professional antigen presenting cell.

15. The method of claim 14, wherein the professional antigen presenting cell is a macrophage or a dendritic cell.

16. The method of any one of claims 6 to 14, wherein the targeting moiety comprises phosphatidylserine.

17. The method of any one of claims 6 to 14, wherein the targeting moiety comprises an antibody or antigen-binding fragment thereof.

18. The method of claim 17, wherein the antibody or antigen-binding fragment thereof is selected from the group consisting of anti-CD163, anti-CD40, anti-CD74, anti-CD206, anti-CD123 antibodies, and antigen-binding fragments thereof.

19. The method of claim 17, wherein the antibody or antigen-binding fragment thereof is selected from the group consisting of anti-DEC205, anti-CD304, anti-CD303, anti-CD40, anti-CD74, anti-BDCA2, and anti-CD123 antibodies, and antigen-binding fragments thereof.

20. The method of any one of claims 6 to 14, wherein the targeting moiety comprises a pathogen-associated molecular pattern (PAMP).

21. The method of any one of claims 6 to 14, wherein the targeting moiety is a mannose cluster or folate.

22. The method of any one of claims 6 to 14, wherein the targeting moiety is a TLR2 agonist.

23. The method of claim 22, wherein the TLR2 agonist is selected from the group consisting of MALP-2 lipoprotein, MALP-404 lipoprotein, outer surface lipoprotein A (OspA), a porin, LcrV, Hsp60, glycoprotein gH/gL, or glycoprotein gB.

24. The method of any one of claims 1 to 23, wherein the supramolecular structure is a lipid nanoparticle.

25. The method of any one of claims 1 to 23, wherein the supramolecular structure is a micelle.

26. The method of any one of claims claim 1 to 23, wherein the supramolecular structure is a liposome.

27. The method of claim 26, wherein the liposome is unilamellar.

28. The method of claim 26, wherein the liposome is multilamellar.

29. The method of any one of claims 1 to 28, wherein the supramolecular structure comprises polydispersity index of from about 0.05 to about 0.3.

30. The method of any one of claims 1 to 29, wherein the supramolecular structure comprises one or more lipids.

31. The method of claim 30, wherein at least one of the one or more lipids is an ionizable lipid.

32. The method of any one of claims 1 to 31, further comprising administering an antibiotic.

33. The method of claim 32, wherein the antibiotic is selected from the group consisting of cephalosporins, carbapenems, penicillins, and fluoroquinolones.

34. The method of claim 32, wherein the antibiotic is selected from the group consisting of thiacetazone, sq-109, bedaquiline, delamanid, pyrazinamide, and isoniazid.

35. The method of claim 32, wherein the antibiotic is selected from the group consisting of azithromycin, clarithromycin, ethambutol, rifampin, and amikacin.

36. The method of any one of claim 1 to 35, wherein the bacteriophage is capable of infecting the bacterial cell.

37. The method of any one of claims 1 to 36, wherein the bacteriophage is a mycobacteriophage.

38. The method of any one of claims 1 to 37, wherein the bacteriophage comprises a polynucleotide encoding a lytic protein.

39. The method of claim 38, wherein the lytic protein is a lysin, an amylase, or a capsule depolymerase.

40. The method of claim 39, wherein:
(a) the lysin is Lysin A or Lysin B; and/or
(b) the amylase is a-amylase or isoamylase.

41. The method of any one of claims 1 to 40, wherein the composition is administered intravenously, orally, topically, or by inhalation.

42. A composition comprising a supramolecular structure comprising a bacteriophage, wherein the supramolecular structure comprises a Z-average mean particle diameter of from about 75 nm to about 750 nm.

43. The composition of claim 42, further comprising a targeting moiety.

44. A composition comprising a supramolecular structure comprising a targeting moiety and a cargo comprising a bacteriophage.

45. The composition of any one of claims 42 to 44, wherein the supramolecular structure comprises a Z-average mean particle diameter of from about 250 nm to about 750 nm.

46. The composition of any one of claims 42 to 44, wherein the supramolecular structure comprises a Z-average mean particle diameter of from about 75 nm to about 250 nm.

47. The composition any one of claims 43 to 46, wherein the targeting moiety is an extracellular targeting moiety targeting a professional antigen presenting cell.

48. The composition of claim 47, wherein the professional antigen presenting cell is a macrophage or a dendritic cell.

49. The composition of any one of claims 43 to 48, wherein the targeting moiety comprises phosphatidylserine.

50. The composition of any one of claims 43 to 48, wherein the targeting moiety comprises an antibody or antigen-binding fragment thereof.

51. The composition of claim 50, wherein the antibody or antigen-binding fragment thereof is selected from the group consisting of anti-CD163, anti-CD40, anti-CD74, anti-CD206, anti-CD123 antibodies, and antigen-binding fragments thereof.

52. The composition of claim 50, wherein the antibody or antigen-binding fragment thereof is selected from the group consisting of anti-DEC205, anti-CD304, anti-CD303, anti-CD40, anti-CD74, anti-BDCA2, and anti-CD123 antibodies, and antigen-binding fragments thereof.

53. The composition of any one of claims 43 to 48, wherein the targeting moiety comprises a PAMP.

54. The composition of any one of claims 43 to 48, wherein the targeting moiety is a mannose cluster or folate.

55. The composition of any one of claims 43 to 48, wherein the targeting moiety is a TLR2 agonist.

56. The composition of claim 55, wherein the TLR2 agonist is selected from the group consisting of MALP-2 lipoprotein, MALP-404 lipoprotein, OspA, a porin, LcrV, Hsp60, glycoprotein gH/gL, or glycoprotein gB.

57. The composition of any one of claims 42 to 56, wherein the supramolecular structure is a lipid nanoparticle.

58. The composition of any one of claims 42 to 56, wherein the supramolecular structure is a micelle.

59. The composition of any one of claims claim 42 to 56, wherein the supramolecular structure is a liposome.

60. The composition of claim 59, wherein the liposome is unilamellar.

61. The composition of claim 59, wherein the liposome is multilamellar.

62. The composition of any one of claims 42 to 61, wherein the supramolecular structure comprises polydispersity index of from about 0.05 to about 0.3.

63. The composition of any one of claims 42 to 62, wherein the supramolecular structure comprises one or more lipids.

64. The composition of claim 63, wherein at least one of the one or more lipids is an ionizable lipid.

65. The composition of any one of claims 41 to 64, further comprising an antibiotic.

66. The composition of claim 65, wherein the antibiotic is selected from the group consisting of cephalosporins, carbapenems, penicillins, and fluoroquinolones.

67. The composition of claim 65, wherein the antibiotic is selected from the group consisting of thiacetazone, sq-109, bedaquiline, delamanid, pyrazinamide, and isoniazid.

68. The composition of claim 65, wherein the antibiotic is selected from the group consisting of azithromycin, clarithromycin, ethambutol, rifampin, and amikacin.

69. The composition of any one of claims 42 to 68, wherein the bacteriophage is capable of infecting a Mycobacterium, Salmonella, Neisseria, Brucella, Escherichia, Listeria, Francisella, Legionella, Yersinia, Staphylococcus, Clostridium, Shigella, or Streptococcus species.

70. The composition of claim 69, wherein:
(a) the Mycobacterium species is M. tuberculosis, M. leprae, M. lepromatosis, M. avium, M. kansasii, M. fortuitum, M. chelonae, M. marinum, or M. abscessus;
(b) the Salmonella species S. enterica, S. typhimurium, or S. bongori;
(c) the Neisseria species is N. gonorrhoeae or N. meningitidis;
(d) the Brucella species is B. melitensis, B. abortus, B. suis, or B. canis;
(e) the Escherichia species is E. coli;
(f) the Listeria species is L. monocytogenes;
(g) the Francisella species is F. tularensis, F. novicida, or F. philomiragia;
(h) the Legionella species L. pneumophila;
(i) the Yersinia species is Y. pestis or Y. enterocolitica;
(j) the Staphylococcus species is S. aureus;
(k) the Clostridium species is C. botulinum, C. perfringens, C. tetani, or C. sordellii;
(l) the Shigella species is S. dysenteriae, S. flexneri, S. boydii, or S. sonnei; or
(m) the Streptococcus species is S. pyogenes, S. agalactiae, S. dysgalactiae, S. bovis, S. anginosus, S. sanguinis, S. mitis, S. mutans, or S. pneumoniae.

71. The composition of any one of claims 42 to 70, wherein the bacteriophage is a mycobacteriophage.

72. The composition of any one of claims 42 to 71, wherein the bacteriophage comprises a polynucleotide encoding a lytic protein.

73. The composition of claim 72, wherein the lytic protein is a lysin, an amylase, or a capsule depolymerase.

74. The composition of claim 73, wherein the lysin is Lysin A or Lysin B.

75. The composition of claim 73 or 74, wherein the amylase is α-amylase or isoamylase.

76. A method of isolating a phage targeted to a bacterium, the method comprising:
contacting a heterogeneous mixture comprising the phage with a detergent, a polar, water-immiscible, aprotic solvent, or a combination thereof to produce a composition comprising a liquid and a solid;
separating the liquid from the solid to produce a supernatant;
concentrating the supernatant to produce an enriched supernatant;
incubating the enriched supernatant with the bacterium to produce a cell mixture comprising the phage, cells, and debris; and
separating the phage from the cells and debris to isolate the phage.

77. The method of claim 76, wherein the heterogeneous mixture is a sewage sludge.

78. The method of claim 76 or 77, wherein the detergent is t-octylphenoxypolyethoxyethanol, polysorbate, or nonoxynol 9.

79. The method of any one of claims 76 to 78, wherein the polar, water-immiscible, aprotic solvent is chloroform.
